(19) United States
(12) Patent Application Publication
(10) Pub. No.: US 2022/0054515 A1
(43) Pub. Date: Feb. 24, 2022

(54) COMPOSITION AND METHOD FOR PROMOTING INTESTINAL BARRIER HEALING

(71) Applicant: Glycom A/S, Hørsholm (DK)

(72) Inventors: Louise Kristine Vigsnæs, Copenhagen NV (DK); Bruce McConnell, La Tour de Peilz (CH); Sami Damak, Lausanne (CH); Francis Foata, La Conversion (CH); Norbert Sprenger, Savigny (CH)

(21) Appl. No.: 17/417,049

(22) PCT Filed: Dec. 19, 2019
(86) PCT No.: PCT/IB2019/061097

§ 371 Date: Jun. 21, 2021

(30) Foreign Application Priority Data
Dec. 21, 2018 (DK) PA201801049

(51) Int. Cl. A61K 31/702; A61K 9/00

(52) U.S. Cl. CPC A61K 31/702; A61K 9/0053

(57) ABSTRACT

A composition and associated packs and methods for (i) promoting gastrointestinal barrier healing in the upper intestinal tract and/or small intestine of a non-infant human suffering from chronic intestinal barrier inflammation, and/or (ii) maintaining remission in the upper intestinal tract and small intestine of a non-infant human suffering from chronic intestinal barrier inflammation. The composition contains an effective amount of a combination of 6′-sialyllactose (6′-SL) and lacto-N-tetraose (LNT).

### FIELD OF THE INVENTION

This invention relates generally to compositions and methods for promoting gastrointestinal barrier healing in the upper intestinal tract and small intestine of non-infant humans.

### BACKGROUND TO THE INVENTION

Inflammation of the gastrointestinal barrier in the upper intestinal tract and small intestine is a common feature of many gastrointestinal conditions. Conditions include inflammation of the oesophagus, coeliac disease, Crohn's Disease (CD) and chemotherapy induced ulceration. In chronic cases, the inflammation leads to damage to the intestinal barrier and the need for intestinal barrier repair.

Inflammation of the oesophagus is often caused by gastrointestinal reflux where stomach contents rise up into the oesophagus. When the condition is chronic, damage to the oesophagus mucosa occurs and complications such as esophagitis, oesophageal stricture, and Barrett's oesophagus arise. When chronic, treatment options include lifestyle changes and medication such as antacids, H2 receptor blockers, proton pump inhibitors, and prokinetics. Prolonged use of these medications has side effects.

Coeliac disease is a chronic autoimmune disorder that primarily affects the small intestine. Coeliac disease is caused by a reaction to gluten, a group of proteins found in wheat and in other grains such as barley and rye. The disease generally occurs in people who are genetically predisposed. Upon exposure to gluten, an abnormal immune response may lead to an inflammatory reaction in the small intestine. This may ultimately result in shortening of the villi lining the small intestine which affects the absorption of nutrients, frequently leading to anaemia. At the present time, the only effective treatment is a strict gluten-free diet. This usually leads to recovery of the intestinal mucosa and reduction in the risk of developing complications. However, the need to adhere to a life-long, gluten-free diet significantly impacts the quality of life of sufferers.

Crohn's disease is a long-term condition that causes inflammation of the lining of the digestive system of both the small and large intestines that ultimately leads to structural damage of the intestinal barrier. It is characterized by alternating periods of remission and relapse. Generally, treatment follows a stepwise approach where the first step is administration of 5-aminosalicylates, which are local acting anti-inflammatories. If the patient fails to respond to 5-aminosalicylates, the second step is often corticosteroids, which tend to provide rapid relief of symptoms and a significant decrease in inflammation (Ford et al. Am. J. Gastroenterol. 106, 590 (2011)). If oral corticosteroid therapy fails, the third step is usually immunomodulators or anti-TNF therapy. Anti-TNF monoclonal antibody therapies are highly effective, at least initially. In particular, the anti-TNF-α antibodies adalimumab, certolizumab pegol, and infliximab are effective for induction of remission.

In general, a major goal is to wean the patient off steroids as soon as possible to prevent long-term adverse effects from these agents. Similarly, patients are exposed to side effects of anti-TNF therapy, such as infections, reactivation of tuberculosis, allergic reactions, skin disorders, demyelinating disorders, and lupus-like autoimmunity. For this reason, these therapies are usually only given to severe patients, or moderate patients with a poor prognosis.

Until recently, most treatments focused on inducing clinical remission and reducing symptoms. However, mucosal healing has recently emerged as a key treatment goal (Neurath et al. Gut 61, 1619 (2012)), particularly in Crohn's disease. Mucosal healing refers to visible resolution of ulceration (Froslie et al. Gastroenterology 133, 412 (2007)). Mucosal healing has been associated with more effective disease control, more frequent steroid-free remission of disease, lower rates of hospitalization and surgery, and improved quality of life as compared with conventional treatment goals. While particularly applicable to Crohn's disease, mucosal healing is applicable to all chronic inflammatory conditions of the intestinal tract.

Due to the side effect profiles of many therapies, attempts have been made to treat patients having inflammatory gastrointestinal conditions with nutritional compositions. Example include nutritional compositions containing casein derived TGF-beta (WO 2014/020004), galactooligosaccharides (WO 2013/016111), human milk oligosaccharides which enhance the expression of mucin-associated proteins (WO 2013/032674), and human milk oligosaccharides for promoting mucosal healing (WO2016/66174). Further human milk oligosaccharides have been proposed for managing conditions such as mood disorders, metabolic issues, and the like which are associated with disrupted intestinal barrier function (WO2017/46711, and WO2017/71716). Most of these approaches using human milk oligosaccharides have inherently focussed on inflammatory conditions of the large intestine because the beneficial effects are mediated through the intestinal microbiota and associated metabolites (e.g. butyrate) or interactions with colonic cells. It is generally accepted that human milk oligosaccharides predominantly modulate the intestinal microbiota in the large intestine.

Therefore, there has remained a need for approaches to promote gastrointestinal barrier healing in the upper intestinal tract and small intestine of non-infant humans suffering from chronic gastrointestinal conditions, which are effective and safe with little or no adverse side effects.

### SUMMARY OF THE INVENTION

In a first aspect, this invention provides 6′-sialyllactose (6′-SL) and/or lacto-N-tetraose (LNT) for use in
promoting gastrointestinal barrier healing in the upper intestinal tract and/or small intestine of a non-infant human suffering from chronic intestinal barrier inflammation, and/or
maintaining remission in the upper intestinal tract and/or small intestine of a non-infant human at risk of a relapse of intestinal barrier dysfunction.

In a second aspect, this invention provides a composition comprising, consisting of or consisting essentially of an effective amount of 6′-sialyllactose (6′-SL) and/or lacto-N-tetraose (LNT) for use in
promoting gastrointestinal barrier healing in the upper intestinal tract and/or small intestine of a non-infant human suffering from chronic intestinal barrier inflammation, and/or
maintaining remission in the upper intestinal tract and/or small intestine of a non-infant human at risk of a relapse of intestinal barrier dysfunction.

The composition may further comprise a source of threonine, serine and/or proline.

In a third aspect, this invention provides a pack for use in:
promoting gastrointestinal barrier healing in the upper intestinal tract and/or small intestine of a non-infant human suffering from chronic intestinal barrier inflammation, and/or
maintaining remission in the upper intestinal tract and small intestine of a non-infant human at risk of a relapse of intestinal barrier dysfunction,

the pack comprising at least 14 individual daily doses of an effective amount of 6′-sialyllactose (6′-SL), lacto-N-tetraose (LNT) or combination thereof.

The pack preferably comprises at least about 21 individual daily doses, for example about 28 daily doses.

A fourth aspect of the invention is a use of
6′-sialyllactose (6′-SL), lacto-N-tetraose (LNT) or combination thereof,
a synthetic composition comprising, consisting of or consisting essentially of 6′-sialyllactose (6′-SL), lacto-N-tetraose (LNT) or combination thereof, or
a pack comprising at least 14 individual daily doses of an effective amount of 6′-sialyllactose (6′-SL), lacto-N-tetraose (LNT) or combination thereof

in the dietary management of a non-infant human suffering from chronic intestinal barrier inflammation.

In a fifth aspect, this invention provides a method for promoting gastrointestinal barrier healing in the upper intestinal tract and/or small intestine of a non-infant human suffering from chronic intestinal barrier inflammation, the method comprising administering to the non-infant human an effective amount of 6′-sialyllactose (6′-SL), lacto-N-tetraose (LNT) or a combination thereof.

In a sixth aspect, this invention provides a method for maintaining remission in the upper intestinal tract and/or small intestine of a non-infant human at risk of a relapse of intestinal barrier dysfunction, the method comprising administering to the non-infant human an effective amount of 6′-sialyllactose (6′-SL), lacto-N-tetraose (LNT) or a combination thereof.

Preferably, the human is administered the combination of 6′-sialyllactose (6′-SL) and lacto-N-tetraose (LNT) for a period of at least 1 week, more preferably for at least 2 weeks. For example, the human can be administered the combination for a period of at least 4 weeks.

The amount of the 6′-sialyllactose (6′-SL) and/or lacto-N-tetraose (LNT) is preferably effective to activate the G protein-coupled receptor 35 (GPR35) in the upper intestinal tract and/or small intestine. In the combination, preferably, the 6′-sialyllactose (6′-SL) and lacto-N-tetraose (LNT) are present in a molar ratio of about 3:1 to about 1:3, for example, about 2:1 to 1:2. Further, the 6′-sialyllactose (6′-SL) and lacto-N-tetraose (LNT) are preferably each present in an amount of 0.1 g to 10 g, more preferably 0.2 g to 7.5 g, for example, 1 g to 5 g.

The non-infant human may be administered a higher dose of 6′-sialyllactose (6′-SL) and/or lacto-N-tetraose (LNT) initially followed by a lower dose of 6′-sialyllactose (6′-SL) and/or lacto-N-tetraose (LNT). The higher dose is preferably about 0.5 g to about 10 g per day (for example about 1 g to about 7.5 g per day) and the lower dose is preferably about 0.1 g to about 5 g per day (for example about 0.2 g to about 3 g per day).

The non-infant human may be suffering from one or more of chronic oesophageal inflammation, coeliac disease, Crohn's Disease (CD), chronic antibiotic induced inflammation and chemotherapy induced ulceration.

### DETAILED DESCRIPTION OF THE INVENTION

It has now been surprisingly found that oral or enteral administration of 6′-sialyllactose (6′-SL) or lacto-N-tetraose (LNT) activates the G protein-coupled receptor 35 (GPR35) and the combination of 6′-sialyllactose (6′-SL) and lacto-N-tetraose (LNT) synergistically activates GPR35. The GPR35 is present throughout the gastrointestinal tract including the upper intestinal tract and/or small intestine. Activation of the GPR35 promotes gastrointestinal barrier healing (Tsukahara et al. Pharmacol. Res. 123, 27 (2017). Further, the 6′-sialyllactose (6′-SL) and lacto-N-tetraose (LNT) are able to access the GPR35s in the upper intestinal tract and/or small intestine.

In this specification, the following terms have the following meanings:

“Bifidobacterium of the B. adolescentis phylogenetic group” means a bacterium selected from the group consisting of Bifidobacterium adolescentis, Bifidobacterium angulatum, Bifidobacterium catenulatum, Bifidobacterium pseudocatenulatum, Bifidobacterium kashiwanohense, Bifidobacterium dentum and Bifidobacterium stercoris (Duranti et al. Appl. Environ. Microbiol. 79, 336 (2013), Bottacini et al. Microbial Cell Fact. 13:S4 (2014)). Preferably, a Bifidobacterium of the B. adolescentis phylogenetic group is Bifidobacterium adolescentis and/or Bifidobacterium pseudocatenulatum.

“Dietary management” means exclusive or partial feeding of patients who, because of a disease, disorder or medical condition are suffering from:
either have a limited, impaired or disturbed capacity to take, digest, absorb, metabolise or excrete ordinary food or certain nutrients contained therein, or metabolites, or
have other medically-determined nutrient requirements

(see: Commission Notice on the classification of Food for Special Medical Purposes of the European Commission, Official Journal of the European Union C 401, 25 Nov. 2017, p. 10-11).

“Enteral administration” means any conventional form for delivery of a composition to a human that causes the deposition of the composition in the gastrointestinal tract (including the stomach). Methods of enteral administration include feeding through a naso-gastric tube or jejunum tube, oral, sublingual and rectal.

“Effective amount” means an amount of a composition that provides a HMO in a sufficient amount to render a desired treatment outcome in a human. An effective amount can be administered in one or more doses to achieve the desired treatment outcome.

“FODMAP” means fermentable oligosaccharides, disaccharides, monosaccharides and polyols.

“Gastrointestinal barrier healing” means one or more of the following:
Improved gastrointestinal barrier repair, such as recovery or repair of the integrity of the gastrointestinal barrier, reduction of permeability upon inflammatory challenge of the gastrointestinal mucosa, and mucosal repair.
Improved barrier structure, such as strengthening of the gastrointestinal barrier, integrity of the gastrointestinal barrier, tight junction structure, and intestinal epithelial lining integrity.
Improved barrier function, such as improvement of gastrointestinal barrier resistance, reduction of gastrointestinal barrier permeability.
Improved barrier protection such as prevention of barrier dysfunction, prevention of barrier leakiness, protection of tight junction structure, protection of the intestinal epithelial lining integrity

“Human milk oligosaccharide” or “HMO” means a complex carbohydrate found in human breast milk (Urashima et al.: Milk Oligosaccharides. Nova Science Publisher (2011); Chen Adv. Carbohydr. Chem. Biochem. 72, 113 (2015)). The HMOs have a core structure comprising a lactose unit at the reducing end that can be elongated by one or more β-N-acetyl-lactosaminyl and/or one or β-more lacto-N-biosyl units, and which core structure can be substituted by an a L-fucopyranosyl and/or an α-N-acetyl-neuraminyl (sialyl) moiety. In this regard, the non-acidic (or neutral) HMOs are devoid of a sialyl residue, and the acidic HMOs have at least one sialyl residue in their structure. The non-acidic (or neutral) HMOs can be fucosylated or non-fucosylated. Examples of such neutral non-fucosylated HMOs include lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH). Examples of neutral fucosylated HMOs include 2′-fucosyllactose (2′-FL), lacto-N-fucopentaose I (LNFP-I), lacto-N-difucohexaose I (LNDFH-I), 3-fucosyllactose (3-FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-fucopentaose VI (LNFP-VI), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (FpLNnH II) and fucosyl-lacto-N-neohexaose (FLNnH). Examples of acidic HMOs include 3′-sialyllactose (3′-SL), 6′-sialyllactose (6′-SL), 3-fucosyl-3′-sialyllactose (FSL), LST a, fucosyl-LST a (FLST a), LST b, fucosyl-LST b (FLST b), LST c, fucosyl-LST c (FLST c), sialyl-LNH (SLNH), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT).

“Microbiota”, “microflora” and “microbiome” mean a community of living microorganisms that typically inhabits a bodily organ or part, particularly the gastro-intestinal organs of humans. The most dominant members of the gastrointestinal microbiota include microorganisms of the phyla of Firmicutes, Bacteroidetes, Actinobacteria, Proteobacteria, Synergistetes, Verrucomicrobia, Fusobacteria, and Euryarchaeota, at genus level Bacteroides, Faecalibacterium, Bifidobacterium, Roseburia, Alistipes, Collinsella, Blautia, Coprococcus, Ruminococcus, Eubacterium and Dorea, at species level Bacteroides uniformis, Alistipes putredinis, Parabacteroides merdae, Ruminococcus bromii, Dorea longicatena, Bacteroides caccae, Bacteroides thetaiotaomicron, Eubacterium hallii, Ruminococcus torques, Faecalibacterium prausnitzii, Ruminococcus lactaris, Collinsella aerofaciens, Dorea formicigenerans, Bacteroides vulgatus and Roseburia intestinalis. The gastrointestinal microbiota includes the mucosa-associated microbiota, which is located in or attached to the mucous layer covering the epithelium of the gastrointestinal tract, and luminal-associated microbiota, which is found in the lumen of the gastrointestinal tract.

“Modulating of microbiota” means exerting a modifying or controlling influence on microbiota, for example an influence leading to an increase in the indigenous intestinal abundance of Bifidobacterium, Barnesiella, Faecalibacterium and/or butyrate producing bacteria. In another example, the influence may lead to a reduction of the intestinal abundance of Ruminococcus gnavus and/or Proteobacteria. “Proteobacteria” are a phylum of Gram-negative bacteria and include a wide variety of pathogenic bacteria, such as Escherichia, Salmonella, Vibrio, Helicobacter, Yersinia and many other notable genera.

“Non-infant human” or “non-infant” means a human of 3 years of age and older. A non-infant human can be a child, a teenager, an adult or an elderly person.

“Oral administration” means any conventional form for the delivery of a composition to a human through the mouth. Accordingly, oral administration is a form of enteral administration.

“Preventive treatment” or “prevention” means treatment given or action taken to diminish the risk of onset or recurrence of a disease.

“Secondary prevention” means prevention of onset of the condition in a high-risk patient, or prevention of reoccurrence of symptoms in a patient who has already has the condition. A “high-risk” patient is an individual who is predisposed to developing the condition, for example, a person with a family history of the condition

“Synthetic composition” means a composition which is artificially prepared and preferably means a composition containing at least one compound that is produced ex vivo chemically and/or biologically, e.g. by means of chemical reaction, enzymatic reaction or recombinantly The synthetic composition typically comprises one or more HMOs. Also, in some embodiments, the synthetic compositions may comprise one or more nutritionally or pharmaceutically active components which do not affect adversely the efficacy of the HMOs. Some non-limiting embodiments of a synthetic composition of the invention are described below.

“Therapy” means treatment given or action taken to reduce or eliminate symptoms of a disease or pathological condition.

“Treat” means to address a medical condition or disease with the objective of improving or stabilising an outcome in the person being treated or addressing an underlying nutritional need. Treat therefore includes the dietary or nutritional management of the medical condition or disease by addressing nutritional needs of the person being treated. “Treating” and “treatment” have grammatically corresponding meanings.

The 6′-sialyllactose (6′-SL) and lacto-N-tetraose (LNT) can be isolated or enriched by well-known processes from milk(s) secreted by mammals including, but not limited to human, bovine, ovine, porcine, or caprine species. They can also be produced by well-known processes using microbial fermentation, enzymatic processes, chemical synthesis, or combinations of these technologies. As examples, using chemistry LNT can be synthesized as described in WO 2012/155916 and WO 2013/044928, a mixture of LNT and LNnT can be made as described in WO 2013/091660, 6′-SL and salts thereof can be made as described in WO 2010/100979, and sialylated oligosaccharides can be made as described in WO 2012/113404. As examples of enzymatic production, sialylated oligosaccharides can be made as described in WO 2012/007588. Biotechnological methods which describe how to make core (non-fucosylated neutral) human milk oligosaccharides optionally substituted by fucose or sialic acid using genetically modified E. coli con be found in WO 01/04341 and WO 2007/101862.

The 6′-sialyllactose (6′-SL) and/or lacto-N-tetraose (LNT) may be complemented with other human milk oligosaccharides. For example, the 6′-SL and/or LNT may be complemented with a fucosylated HMO selected from 2′-FL, 3-FL and DFL.

In all aspects of the invention, a preferred embodiment is a combination (mixture) of 6′-sialyllactose (6′-SL) and lacto-N-tetraose (LNT), more preferably a combination (mixture) consisting of or consisting essentially of 6′-SL and LNT.

The 6′-sialyllactose (6′-SL) and/or lacto-N-tetraose (LNT) can be used as it is or they are (neat), without any carrier and/or diluent. In other embodiment, the 6′-sialyllactose (6′-SL) and/or lacto-N-tetraose (LNT) is/are used in a synthetic composition with one or more inert carriers/diluents that are acceptable in nutritional or pharmaceutical compositions, for example solvents (e.g. water, water/ethanol, oil, water/oil), dispersants, coatings, absorption promoting agents, controlled release agents, inert excipients (e.g. starches, polyols, granulating agents, microcrystalline cellulose, diluents, lubricants, binders, disintegrating agents). These compositions do not contain prebiotic and/or probiotic [that is: a synthetic composition consisting of a) 6′-sialyllactose (6′-SL) and/or lacto-N-tetraose (LNT), b) one or more inert carriers/diluents that are acceptable in nutritional or pharmaceutical compositions]. In other embodiment, the 6′-sialyllactose (6′-SL) and/or lacto-N-tetraose (LNT) is/are used in a synthetic pharmaceutical or nutritional composition that may contain a prebiotic and/or probiotic.

In this regard, the following embodiments for use in promoting gastrointestinal barrier healing in the upper intestinal tract and/or small intestine of a non-infant human suffering from chronic intestinal barrier inflammation, and/or maintaining remission in the upper intestinal tract and/or small intestine of a non-infant human at risk of a relapse of intestinal barrier dysfunction are especially comprised:
a single HMO which is 6′-SL,
a single HMO which is LNT,
exactly two HMOs which are 6′-SL and LNT (a mixture of HMOs consisting of or consisting essentially of 6′-SL and LNT),
6′-SL and at least a further HMO different than LNT,
LNT and at least a further HMO different than 6′-SL,
6′-SL, LNT and at least a further HMO different than 6′-SL and LNT,
a synthetic composition comprising, consisting of or consisting essentially of 6′-SL,
a synthetic composition comprising, consisting of or consisting essentially of LNT,
a synthetic composition comprising, consisting of or consisting essentially of 6′-SL and LNT,
a synthetic composition comprising, consisting of or consisting essentially of 6′-SL and at least a further HMO different than LNT,
a synthetic composition comprising, consisting of or consisting essentially of LNT and at least a further HMO different than 6′-SL,
a synthetic composition comprising, consisting of or consisting essentially of 6′-SL, LNT and at least a further HMO different than 6′-SL and LNT.

Furthermore, the preferred embodiments of the daily doses comprised in the pack according to the third aspect are those disclosed above.

Furthermore, the preferred embodiments of the use in the dietary management of a non-infant human suffering from chronic intestinal barrier inflammation according to the fourth aspect are those disclosed above.

Furthermore, the preferred embodiments of the methods according to any of the fifth or sixth aspects are those disclosed above.

The synthetic composition can be in the form of a nutritional composition. For example, the nutritional composition can be a food composition, a rehydration solution, a medical food or food for special medical purposes, a nutritional supplement and the like. The nutritional composition can contain sources of protein, lipids and/or digestible carbohydrates and can be in powdered or liquid forms. The composition can be designed to be the sole source of nutrition or as a nutritional supplement.

Suitable protein sources include milk proteins, soy protein, rice protein, pea protein and oat protein, or mixtures thereof. Milk proteins can be in the form of milk protein concentrates, milk protein isolates, whey protein or casein, or mixtures of both. The protein can be whole protein or hydrolysed protein, either partially hydrolysed or extensively hydrolysed. Hydrolysed protein offers the advantage of easier digestion which can be important for humans with inflamed or compromised GI tracts. The protein can also be provided in the form of free amino acids. The protein can comprise about 5% to about 30% of the energy of the nutritional composition, normally about 10% to 20%.

The protein source can be a source of glutamine, threonine, cysteine, serine, proline, or a combination of these amino acids. The glutamine source can be a glutamine dipeptide and/or a glutamine enriched protein. Glutamine can be included due to the use of glutamine by enterocytes as an energy source. Threonine, serine and proline are important amino acids for the production of mucin. Mucin coats the gastrointestinal tract and can improve intestinal barrier function and mucosal healing. Cysteine is a major precursor of glutathione, which is key for the antioxidant defences of the body.

Suitable digestible carbohydrates include maltodextrin, hydrolysed or modified starch or corn starch, glucose polymers, corn syrup, corn syrup solids, high fructose corn syrup, rice-derived carbohydrates, pea-derived carbohydrates, potato-derived carbohydrates, tapioca, sucrose, glucose, fructose, sucrose, lactose, honey, sugar alcohols (e.g. maltitol, erythritol, sorbitol), or mixtures thereof. Preferably the composition is reduced in or free from added lactose or other FODMAP carbohydrates. Generally digestible carbohydrates provide about 35% to about 55% of the energy of the nutritional composition. A particularly suitable digestible carbohydrate is a low dextrose equivalent (DE) maltodextrin.

Suitable lipids include medium chain triglycerides (MCT) and long chain triglycerides (LCT). Preferably the lipid is a mixture of MCTs and LCTs. For example, MCTs can comprise about 30% to about 70% by weight of the lipids, more specifically about 50% to about 60% by weight. MCTs offer the advantage of easier digestion which can be important for humans with inflamed or compromised GI tracts. Generally, the lipids provide about 35% to about 50% of the energy of the nutritional composition. The lipids can contain essential fatty acids (omega-3 and omega-6 fatty acids). Preferably these polyunsaturated fatty acids provide less than about 30% of total energy of the lipid source.

Suitable sources of long chain triglycerides are rapeseed oil, sunflower seed oil, palm oil, soy oil, milk fat, corn oil, high oleic oils, and soy lecithin. Fractionated coconut oils are a suitable source of medium chain triglycerides. The lipid profile of the nutritional composition is preferably designed to have a polyunsaturated fatty acid omega-6 (n-6) to omega-3 (n-3) ratio of about 4:1 to about 10:1. For example, the n-6 to n-3 fatty acid ratio can be about 6:1 to about 9:1.

The nutritional composition may also include vitamins and minerals. If the nutritional composition is intended to be a sole source of nutrition, it preferably includes a complete vitamin and mineral profile. Examples of vitamins include vitamins A, B-complex (such as B1, B2, B6 and B12), C, D, E and K, niacin and acid vitamins such as pantothenic acid, folic acid and biotin. Examples of minerals include calcium, iron, zinc, magnesium, iodine, copper, phosphorus, manganese, potassium, chromium, molybdenum, selenium, nickel, tin, silicon, vanadium and boron.

The nutritional composition can also include a carotenoid such as lutein, lycopene, zeaxanthin, and beta-carotene. The total amount of carotenoid included can vary from about 0.001 μg/ml to about 10 μg/ml. Lutein can be included in an amount of from about 0.001 μg/ml to about 10 μg/ml, preferably from about 0.044 μg/ml to about 5 μg/ml of lutein. Lycopene can be included in an amount from about 0.001 μg/ml to about 10 μg/ml, preferably about 0.0185 μg/ml to about 5 μg/ml of lycopene. Beta-carotene can comprise from about 0.001 μg/ml to about 10 mg/ml, for example about 0.034 μg/ml to about 5 μg/ml of beta-carotene.

The nutritional composition preferably also contains reduced concentrations of sodium, for example, from about 300 mg/l to about 400 mg/l. The remaining electrolytes can be present in concentrations set to meet needs without providing an undue renal solute burden on kidney function. For example, potassium is preferably present in a range of about 1180 to about 1300 mg/l, and chloride is preferably present in a range of about 680 to about 800 mg/l.

The nutritional composition can also contain various other conventional ingredients such as preservatives, emulsifying agents, thickening agents, buffers, fibres and prebiotics (e.g. fructooligosaccharides, galactooligosaccharides), probiotics (e.g. B. animalis subsp. lactis BB-12, B. lactis HN019, B. lactis Bi07, B. infantis ATCC 15697, L. rhamnosus GG, L. rhamnosus HNOOI, L. acidophilus LA-5, L. acidophilus NCFM, L. fermentum CECT5716, B. longum BB536, B. longum AH1205, B. longum AH1206, B. breve M-16V, L. reuteri ATCC 55730, L. reuteri ATCC PTA-6485, L. reuteri DSM 17938), antioxidant/anti-inflammatory compounds including tocopherols, carotenoids, ascorbate/vitamin C, ascorbyl palmitate, polyphenols, glutathione, and superoxide dismutase (melon), other bioactive factors (e.g. growth hormones, cytokines, TFG-β), colorants, flavours, and stabilisers, lubricants, and so forth.

The nutritional composition is preferably free from gluten.

The nutritional composition can be formulated as a soluble powder, a liquid concentrate, or a ready-to-use formulation. The composition can be fed to a human in need via a nasogastric tube or orally. Various flavours, fibres and other additives can also be present.

The nutritional compositions can be prepared by any commonly used manufacturing techniques for preparing nutritional compositions in solid or liquid form. For example, the composition can be prepared by combining various feed solutions. A protein-in-fat feed solution can be prepared by heating and mixing the lipid source and then adding an emulsifier (e.g. lecithin), fat soluble vitamins, and at least a portion of the protein source while heating and stirring. A carbohydrate feed solution is then prepared by adding minerals, trace and ultra-trace minerals, thickening or suspending agents to water while heating and stirring. The resulting solution is held for 10 minutes with continued heat and agitation before adding carbohydrates (e.g. the HMOs including 6′-sialyllactose (6′-SL) and/or lacto-N-tetraose (LNT) and digestible carbohydrate sources). The resulting feed solutions are then blended together while heating and agitating and the pH adjusted to 6.6-7.0, after which the composition is subjected to high-temperature short-time processing during which the composition is heat treated, emulsified and homogenized, and then allowed to cool. Water soluble vitamins and ascorbic acid are added, the pH is adjusted to the desired range if necessary, flavours are added, and water is added to achieve the desired total solid level.

For a liquid product, the resulting solution can then be aseptically packed to form an aseptically packaged nutritional composition. In this form, the nutritional composition can be in ready-to-feed or concentrated liquid form. Alternatively, the composition can be spray-dried and processed and packaged as a reconstitutable powder.

When the nutritional product is a ready-to-feed nutritional liquid, it may be preferred that the total concentration of 6′-sialyllactose (6′-SL) and/or lacto-N-tetraose (LNT) in the liquid, by weight of the liquid, is from about 0.1% to about 1.5%, including from about 0.2% to about 1.0%, for example from about 0.3% to about 0.7%. When the nutritional product is a concentrated nutritional liquid, it may be preferred that the total concentration of 6′-sialyllactose (6′-SL) and/or lacto-N-tetraose (LNT) in the liquid, by weight of the liquid, is from about 0.2% to about 3.0%, including from about 0.4% to about 2.0%, for example from about 0.6% to about 1.5%.

In another embodiment, the nutritional composition is in a unit dosage form. The unit dosage form can contain an acceptable food-grade carrier, e.g. phosphate buffered saline solution, mixtures of ethanol in water, water and emulsions such as an oil/water or water/oil emulsion, as well as various wetting agents or excipients. The unit dosage form can also contain other materials that do not produce an adverse, allergic or otherwise unwanted reaction when administered to a human. The carriers and other materials can include solvents, dispersants, coatings, absorption promoting agents, controlled release agents, and one or more inert excipients, such as starches, granulating agents, microcrystalline cellulose, diluents, lubricants, binders, and disintegrating agents. Preferably carriers and other materials are low in FODMAPs or contain no FODMAPs. Preferably, the unit dosage form comprises primarily 6′-sialyllactose (6′-SL) and/or lacto-N-tetraose (LNT) with a minimum amount of binders and/or excipients. Unit dosage forms are particularly suitable when nutritionally incomplete or not intended as a sole source of nutrition.

A unit dosage form of this invention can be administered orally, e.g. as a tablet, capsule, or pellet containing a predetermined amount of the mixture, or as a powder or granules containing a predetermined concentration of the mixture or a gel, paste, solution, suspension, emulsion, syrup, bolus, electuary, or slurry, in an aqueous or non-aqueous liquid, containing a predetermined concentration of the mixture. An orally administered composition can include one or more binders, lubricants, inert diluents, flavouring agents, and humectants. An orally administered composition such as a tablet can optionally be coated and can be formulated to provide sustained or controlled release of the 6′-SL and/or LNT.

A unit dosage form of this invention can also be administered by naso-gastric tube or direct infusion into the GI tract or stomach.

A unit dosage form of this invention can also include therapeutic agents such as antibiotics, probiotics, analgesics, and anti-inflammatory agents.

The proper dosage of such a composition for a human can be determined in a conventional manner, based upon factors such as the human's condition, immune status, body weight and age. In some cases, the dosage will be at a concentration similar to that found for the 6′-SL and/or LNT of the composition in human breast milk. The required amount would generally be in the range from about 0.1 g to about 10 g per day, in certain embodiments from about 0.2 g to about 7.5 g per day, for example about 1 g to about 5 g per day. Appropriate dose regimes can be determined by methods known to those skilled in the art.

In further embodiment, the 6′-sialyllactose (6′-SL) and/or lacto-N-tetraose (LNT) can be formulated as a pharmaceutical composition. The pharmaceutical composition can contain a pharmaceutically acceptable carrier, e.g. phosphate buffered saline solution, mixtures of ethanol in water, water and emulsions such as an oil/water or water/oil emulsion, as well as various wetting agents or excipients. The pharmaceutical composition can also contain other materials that do not produce an adverse, allergic or otherwise unwanted reaction when administered to humans. The carriers and other materials can include solvents, dispersants, coatings, absorption promoting agents, controlled release agents, and one or more inert excipients, such as starches, granulating agents, microcrystalline cellulose, diluents, lubricants, binders, and disintegrating agents. Preferably carriers and other materials are low in FODMAPs or contain no FODMAPs.

The pharmaceutical compositions can be administered orally, e.g. as a tablet, capsule, or pellet containing a predetermined amount, or as a powder or granules containing a predetermined concentration or a gel, paste, solution, suspension, emulsion, syrup, bolus, electuary, or slurry, in an aqueous or non-aqueous liquid, containing a predetermined concentration. Orally administered compositions can include binders, lubricants, inert diluents, flavouring agents, and humectants. Orally administered compositions such as tablets can optionally be coated and can be formulated to provide sustained, delayed or controlled release of the mixture therein.

The pharmaceutical compositions can also be administered by rectal suppository, aerosol tube, naso-gastric tube or direct infusion into the GI tract or stomach.

The pharmaceutical compositions can also include therapeutic agents such as antibiotics, probiotics, analgesics, and anti-inflammatory agents.

The proper dosage of these compositions for a human can be determined in a conventional manner, based upon factors such condition, immune status, body weight and age. In some cases, the dosage will be at a concentration similar to that found for the 6′-sialyllactose (6′-SL) and/or lacto-N-tetraose (LNT) in human breast milk. The required amount would generally be in the range from about 0.1 g to about 10 g per day, in certain embodiments from about 0.2 g to about 7.5 g per day, for example from about 1 g to about 5 g per day. Appropriate dose regimes can be determined by conventional methods.

The amount of 6′-SL and/or LNT required to be administered for (i) promoting gastrointestinal barrier healing in the upper intestinal tract and/or small intestine of a non-infant human suffering from chronic intestinal barrier inflammation, and/or (ii) maintaining remission in the upper intestinal tract and small intestine of a non-infant human suffering from chronic intestinal barrier inflammation, will vary depending upon factors such as the risk and severity of the underlying condition, any other medical conditions or diseases, age, the form of the composition, and other medications being administered. Further the amount may vary depending upon whether the combination is being used to deliver a direct effect (when the dose may be higher) or whether the combination is being used as a secondary prevention/maintenance (when the dose may be lower). However, the required amount can be readily set by a medical practitioner and would generally be in the range from about 0.1 g to about 10 g per day, in certain embodiments from about 0.2 g to about 7.5 g per day, for example from about 1 g to about 5 g per day. An appropriate dose can be determined based on several factors, including, for example, body weight and/or condition, the severity of the underlying condition being treated or prevented, other ailments and/or diseases, the incidence and/or severity of side effects and the manner of administration. Appropriate dose ranges may be determined by methods known to those skilled in the art. During an initial treatment phase, the dosing can be higher (for example 0.5 g to 10 g per day, preferably 1 g to 7.5 g per day). During a maintenance phase, the dosing can be reduced (for example, 0.1 g to 5 g per day, preferably 0.2 g to 3 g per day).

### EXAMPLES

Examples Are Now Described to Further Illustrate the Invention

### Example 1—GPR35 Activation Model

Six human milk oligosaccharides (LNnT, LNT, 2′-FL, DFL, 6′-SL and 3′-SL) were screened for their ability to activate the GPR35 receptor in an in vitro assay. The screening was conducted by DiscoverX (Fremont, Calif., USA) using the PathHunter β-Arresin assay. This assay monitored the activation of GPR35 expressed in cultured cells, using Enzyme Fragment Complementation with β-galactosidase (β-Gal) as the functional reporter. The enzyme was split into two inactive complementary portions, EA and PK, expressed as fusion proteins in the cell. EA was fused to β-Arrestin and PK was fused to GPR35. When the GPR35 is activated and β-Arrestin is recruited to the receptor, PK and EA complementation occurs, restoring β-Gal activity, which was measured using chemiluminescent Detection Reagents.

The GPR35 cell line was expanded from frozen stocks and cultured according to standard procedures. Cells were seeded in a total volume of 20 μl into white walled, 384-well microplates and incubated at 37° C. for the appropriate time prior to testing. All of the testings were done in duplicate.

HMO samples were shipped to DiscoverX as concentrated stocks in Phosphate Buffered saline. On the test day, a 5× compound working intermediate was prepared in PBS. Five μl of 5× sample were added to cells and incubated at room temperature for 3 to 5 hours.

Assay signal was generated through a single addition of 12.5 μl (50 v/v %) of PathHunter Detection reagent cocktail, followed by one-hour incubation at room temperature. Microplates were read following signal generation with a PerkinElmer Envision™ instrument for chemiluminescent signal detection. Zaprinast (which is known to be an activator of GPR35) was used as positive control. Dose-responses were generated by iterative 3× dilutions of the highest concentration tested (10 mM).

LNT activated GPR35 with an EC50 of 4.5 mM. 6′-SL activated GPR35 with an EC50 of 6.5 mM. No activation of GPR35 was obtained with 3′-SL, 2′-FL, DFL and LNnT. LNT and 6′-SL were then tested in combination. An equimolar mix of 6′-SL and LNT activated GPR35 with an EC50 of 2.2 mM. This result demonstrates a synergy between 6′-SL and LNT.

A mixture of all six HMOs at an equimolar ratio was also tested, showing results similar to the combination of LNT and 6′-SL. These results demonstrated that the synergistic effect of LNT and 6′-SL was maintained even when mixed with other human milk oligosaccharides.

### Example 2—Nutritional Composition

A ready to feed nutritional composition is prepared from water, maltodextrin, enzymatically hydrolysed whey protein (from cows milk), medium chain triglycerides (from coconut and/or palm kernel oil), corn-starch, soybean oil, soy lecithin, 6′-SL, LNT, magnesium chloride, calcium phosphate, guar gum, sodium ascorbate, potassium citrate, sodium phosphate, calcium citrate, choline chloride, potassium chloride, sodium citrate, magnesium oxide, taurine, L-carnitine, alpha-tocopheryl acetate, zinc sulphate, ferrous sulphate, niacinamide, calcium pantothenate, vitamin A palmitate, citric acid, manganese sulphate, pyridoxine hydrochloride, vitamin D3, copper sulphate, thiamine mononitrate, riboflavin, beta carotene, folic acid, biotin, potassium iodide, chromium chloride, sodium selenate, sodium molybdate, phytonadione, vitamin B12.

The composition has a calorific density of 1.0 kcal/ml with a caloric distribution (% of kcal) as follows: protein: 16%, carbohydrate: 51%, fat: 33%. The protein source has an NPC:N ratio of 131:1. The MCT:LCT ratio is 70:30 and the n6:n3 ratio is 7.4:1. The osmolality (mOsm/kg water) is 270 when unflavoured. The composition contains 85% water and 1500 ml meets 100% of the RDI for 22 key micronutrients.

The composition has a balanced peptide profile which promotes GI absorption and integrity and the enzymatically hydrolysed 100% whey protein facilitates gastric emptying. The MCT decreases the potential for fat malabsorption. The composition is nutritionally complete for tube feeding or oral supplementation.

### Example 3—Tablet Composition

A tablet is prepared from 6′-SL, LNT, hydroxypropyl methylcellulose, sodium alginate, gum, microcrystalline cellulose, colloidal silicon dioxide, and magnesium stearate. All raw materials except the magnesium stearate are placed into a high shear granulator and premixed. Water is sprayed onto the premix while continuing to mix at 300 rpm. The granulate is transferred to a fluidised bed drier and dried at 75° C. The dried powder is sieved and sized using a mill. The resulting powder is then lubricated with magnesium stearate and pressed into tablets. The tablets each contain 325 mg of the combination of 6′-SL and LNT. The tablets each have a weight of 750 mg.

### Example 4—Capsule Composition

A capsule is prepared by filling about 1 g of a combination of 6′-SL and LNT into a 000-gelatine capsule using a filing machine. The capsules are then closed. The 6′-SL and LNT are in free flowing, powder form.

### Example 5—Nutritional Composition

A combination of 6′-SL and LNT are introduced into a rotary blender in a 1:1 molar ratio. An amount of 0.25 w % of silicon dioxide is introduced into the blender and the mixture blended for 10 minutes. The mixture is then agglomerated in a fluidised bed and filled into 5 gram stick packs and the packs are sealed.

1-21 (canceled)

22. A method comprising
selecting a non-infant human experiencing oesophageal inflammation;
selecting an initial dosage of a composition comprising an effective amount of one or two synthetic human milk oligosaccharides (HMOs) chosen from the group consisting of 6′-sialyllactose (6′-SL), lacto-N-tetraose (LNT) and a mixture thereof, the selected amount effective to induce direct activation of G protein-coupled receptor 35 (GPR35) in the oesophagus of the non-infant human; and
promoting GPR35 mediated mucosal healing in the oesophagus of the non-infant human experiencing the oesophageal inflammation by administering the initial dosage of the composition during an initial treatment phase.

23. The method of claim 22, wherein the molar ratio of the 6′-SL to the LNT in the mixture is from 2:1 to 1:2.

24. The method of claim 22, wherein the selected effective amount of the mixture of the 6′-SL and LNT induces a synergistic effect in the GPR35 activity relative to sum of the GPR35 activity induced by the 6′-SL alone and the LNT alone.

25. The method of claim 24, further comprising administering one or more additional HMOs with the mixture of the 6′-SL and LNT.

26. The method of claim 25, wherein the synergistic effect of the 6′-SL and LNT is maintained with the administering of the one or more additional HMOs with the mixture of the 6′-SL and LNT.

27. The method of claim 22, wherein the initial dosage of the chosen HMOs in the composition is from about 1 g to about 7.5 g per day.

28. The method of claim 22, further comprising administering to the non-infant human during a maintenance phase, a maintenance dosage of the chosen HMOs of from 0.2 g to about 3 g per day.

29. The method of claim 22, further comprising reducing the likelihood of the non-infant human experiencing one or more oesophageal disorders selected from esophagitis, oesophageal stricture, and Barrett's oesophagus, by administering the initial dosage of the composition during the initial treatment phase.

30. The method of claim 29, wherein reducing the likelihood of the non-infant human experiencing the one or more oesophageal disorders comprises reducing the likelihood of the non-infant human experiencing chemotherapy-induced oesophageal ulceration.

31. The method of claim 29, the oesophageal inflammation in the upper gastrointestinal (GI) tract and/or the small intestine is reduced without administering to the non-infant human, treatments selected from antacids, H2 receptor blockers, proton pump inhibitors, prokinetics, and combinations thereof, during the initial treatment phase.

32. A method comprising:
selecting a non-infant human experiencing inflammation in the small intestine associated with Crohn's disease;
selecting an initial dosage of a composition comprising an effective amount of one or two synthetic human milk oligosaccharides (HMOs) chosen from the group consisting of 6′-sialyllactose (6′-SL), lacto-N-tetraose (LNT) and a mixture thereof, the selected amount effective to induce direct activation of G protein-coupled receptor 35 (GPR35) in the small intestine of the non-infant human; and
promoting GPR35 mediated mucosal healing in the small intestine of the non-infant human by administering the initial dosage of the composition during an initial treatment phase.

33. The method of claim 32, wherein the molar ratio of the 6′-SL to the LNT in the mixture is from 2:1 to 1:2.

34. The method of claim 33, wherein the selected effective amount of the mixture of the 6′-SL and LNT induces a synergistic effect in the GPR35 activity relative to sum of the GPR35 activity induced by the 6′-SL alone and the LNT alone.

35. The method of claim 34, further comprising administering one or more additional HMOs with the mixture of the 6′-SL and LNT.

36. The method of claim 35, wherein the synergistic effect of the 6′-SL and LNT is maintained with the administering of the one or more additional HMOs with the mixture of the 6′-SL and LNT.

37. The method of claim 32, wherein the initial dosage of the chosen HMOs in the composition is from about 1 g to about 7.5 g per day.

38. The method of claim 32, further comprising maintain a remission of the inflammation in the small intestine by administering to the non-infant human during a maintenance phase, a maintenance dosage of the chosen HMOs of from 0.2 g to about 3 g per day.

39. The method of claim 32, wherein the inflammation in the small intestine is reduced without administering treatments selected from 5-aminosalicylatessteroids, corticosteroids, anti-tumour necrosis factor (TNF) drugs, and combinations thereof, to the non-infant human during the initial treatment phase.

40. A method comprising:
selecting a non-infant human experiencing inflammation in the small intestine associated with coeliac disease;
selecting an initial dosage of a composition comprising an effective amount of one or two synthetic human milk oligosaccharides (HMOs) chosen from the group consisting of 6′-sialyllactose (6′-SL), lacto-N-tetraose (LNT) and a mixture thereof, the selected amount effective to induce direct activation of G protein-coupled receptor 35 (GPR35) in the upper GI tract or the small intestine of the non-infant human; and
promoting GPR35 mediated mucosal healing in the upper GI tract or the small intestine of the non-infant human by administering the initial dosage of the composition during an initial treatment phase.

41. The method of claim 40, wherein:
the selected effective amount of the mixture of the 6′-SL and LNT induces a synergistic effect in the GPR35 activity relative to sum of the GPR35 activity induced by the 6′-SL alone and the LNT alone; and
inflammation in the upper GI tract or the small intestine is reduced without adherence by the non-infant human to a strict gluten free diet.
